(19) United States
(12) Patent Application Publication
(10) Pub. No.: US 2002/0182696 A1
(43) Pub. Date: Dec. 5, 2002

(54) Stabilized enzyme compositions

(75) Inventors: Luppo Edens, Rotterdam (NL); Luc Van der Heijden, Leiden (NL); Albert Jon Vis, Rotterdam (NL)

Correspondence Address: Leopold Presser, Scully, Scott, Murphy & Presser, 400 Garden City Plaza, Garden City, NY, 11530, US

(73) Assignee: COSMOFERM B.V., Delft (NL)

(21) Appl. No.: 10/132,344

(22) Filed: Apr. 25, 2002

(30) Foreign Application Priority Data
Apr. 27, 2001 (EP) 01201569.9

(51) Int. Cl. C12N 9/96; A61K 38/54

(52) U.S. Cl. 435/188; 424/94.3

(57) ABSTRACT

A stabilized enzyme composition comprising an amount of at least one enzyme, an amount of a polyol for stabilizing the enzyme and an amount of an acrylamide polymer thickening agent, characterized in that as the acrylamide polymer use is made of a —(CH₂CR(CONH₂)— polymer, in which R is either hydrogen or a methyl group. The concentration of the polyacrylamide in the composition varies from 0.05 to 15 wt %.

### DESCRIPTION

1. Field of the Invention

The present invention relates to a composition suitable for topical application comprising an amount of an enzyme, an amount of a polyol for stabilizing the enzyme and amount of an acrylamide polymer thickening agent.

2. Background of the Invention

Topical application of enzymes has found numerous applications in the cosmetic as well as in the pharmaceutical field. Superoxide dismutase and catalase enzymes for example have often been quoted in fighting radicals that are generated upon exposure to UV light, see U.S. Pat. No. 4,129,644. Glycosidases and lysozyme are thought capable of enhancing the process of skin desquamation (see, WO93/19731) or have been used in the treatment of acne (see, Hungarian Application No. 057608). Several patent applications, including WO 94/18945 and JP02204407, disclose the use of the enzyme transglutaminase as well as applications involving lipolytic enzymes to control greasiness of hair (see, for example, DE19824072). Combining topical application of photolyase with light therapy has been reported to repair DNA damage in UV-irradiated human skin (see, for example, PNAS 97, 1790-1795 (2000)). The use of protease enzymes has been suggested to replace α-hydroxy acids in skin peeling formulations (see, for example, JP04027388) and to support actives like retinoic acid in the treatment of acne, e.g. WO/9848775. The commercial potential use of proteases in cosmetic applications to soften and smoothen the skin has been enhanced by recent production process developments that enable the production of hypoallergenic versions of various proteases.

Application of enzymes in consumer products however implies the need of using enzymes having a long-term stability at ambient temperatures in aqueous conditions as the majority of consumer products are water based. A potential problem that one may be confronted with is that active enzymes are inherently labile and show limited shelf stability. To remedy this, a stabilizer is added to the enzyme composition. Polyols are frequently used stabilizing agents, which are generally added in relatively high concentrations to the enzyme composition. Polyol stabilizing agents may be combined with other stabilizers such as metal ions like calcium or reducing agents, the latter are typically used in relatively low concentrations.

The primary role of a polyol in a stabilized enzyme-containing formulation is to lower the water activity (Aw) of the formulation. The Aw is defined as the ratio of the partial pressure of the water vapor at the surface of the composition and the saturation pressure of water vapor above pure water at a certain temperature. Generally speaking, water activities below 0.9 are required to stabilize enzyme-containing formulations. Although other compounds may be used for this purpose, polyols are preferred since they combine various advantages such as a good compatibility with various biological systems and a good solubility in water. The desired long-term stability of enzyme-containing formulations is achieved by maintaining the water activity level rather low. To achieve this, polyol concentrations of up to 40% (v/v) or more have been used. However, the presence of such high polyol concentrations is considered unacceptable in compositions for topical use.

To ensure that the enzyme maintains it's active structure, divalent cations, such as calcium ions, are typically added to an enzyme/polyol system. Low concentrations of divalent cations are generally sufficient to obtain the necessary stabilization. The stabilizing effect is achieved because the divalent cations show a strong bonding to specific binding sites on the surface of the enzyme molecules. A similar stabilization may be obtained with so-called non-specific ion effects, but the ion concentrations required to achieve these effects are much higher. This is undesirable since a high ion concentration is usually incompatible with galenic formulation demands.

Stabilizing the enzyme with a polyol however entails a temporary loss of enzyme activity. The enzyme activity may be restored upon addition of water. The implication of such a technique is that the addition of extra water is essential to reactivate the enzyme in, for example, topical application of the formulation. A dispensing system is disclosed in WO 97/27841 which simultaneously enhances the shelf life of stabilized enzyme compositions having low water activity, while restoring the enzyme activity upon application to the skin. The dispensing system disclosed in WO 97/27841 enables simultaneous delivery of two components of an aqueous composition: a first composition comprising stably formulated enzyme and a second aqueous composition. Upon delivery, both aqueous compositions are mixed, either in situ or in the dispensing system in such a way that a final composition is obtained which contains the enzyme in an active form which is suitable for direct topical use.

When preparing enzyme compositions for personal care, one is confronted with the additional problem that the compositions, besides providing a sufficiently long shelf stability, should be presented to the consumer as attractive formulations that are convenient to use. To achieve this, the enzyme formulation contained in the dispensing system needs to have a certain minimum viscosity, which may be obtained by adding a suitable thickening agent to the enzyme formulation. Unfortunately, many of the thickening agents known in the art appear to be incompatible with enzyme compositions with a low calcium ion concentration and a high polyol concentration. It has often been encountered that the thickening effect is insufficient resulting in a formulation that has an unattractive appearance which is undesirable for cosmetic applications.

European Patent Application 980,170 relates to triple water/oil/water emulsions for cosmetic and dermatological applications, skin cleaning emulsions and, in particular, to formulations providing a controlled release of active agent. The formulations of European Patent Application 980,170 aim at protecting the active agent against external destabilizing compounds, as the active agent is sensitive to water, oxygen and active acids. The formulation disclosed in European Patent Application 980,170 comprises an internal and an external aqueous phase, separated from each other by an oil layer. To prevent destabilization of the external aqueous phase, the formulation contains a gelled external water phase, and an oil phase which, with the internal water phase, forms a primary water/oil emulsion. The oil phase contains an emulsifying polyol. The water phase contains a mixture of a poly (sulfonic acrylamido methyl propane) acid, which captures effective Ca ions from the solution.

From WO 9510605 enzyme stabilizing compositions are known that contain two or more of the following components: (a) a tris (hydroxymethyl) methyl compound, (b) a substituted polyacrylamide as a poly electrolyte, (c) a pH buffer and (d) a polyol additive.

European Patent Application 382,619 teaches to encapsulate an aqueous phase containing an enzyme and a polyacrylamide gelling agent in lipidic niosomes. The niosomes, in turn, are mixed into an aqueous dispersion comprising a liquid oil phase that is not miscible with water.

European Patent Application 750,905 relates to a patch for improving the topical circulatory dynamics and metabolism and, if necessary, exerting medicinal effects on a painful, stiff neck and/or shoulder, while giving favorable warm-bathing effects. The patch is gradually dissolved during bathing. To achieve these effects, the patch comprises a water-soluble adhesive sheet and a water-soluble protective material laminated thereon. The water-soluble adhesive sheet contains water and a water-soluble polymer as an adhesive agent, for example, a polydimethylacrylamide, (meth)acrylate or (meth)acrylamide polymers having a dialkylamino group. The patch may contain an enzyme. However, the polyol that may be present on the adhesive sheet is added to achieve a plasticising effect.

### SUMMARY OF THE INVENTION

An object of the present invention is to provide a stable enzyme composition having a sufficiently high viscosity and a cosmetically attractive appearance.

This is achieved in the present invention by providing a composition containing, as a polyacrylamide thickener, a polymer of —(CH₂CR(CONH₂)— units, in which R may be H, or CH₃. Polyacrylamide compounds suitable for use with the present invention thus include acrylamide as well as methacrylamide polymers.

It has now been found that the incorporation of the above described polyacrylamide thickening agent allows increasing the viscosity of a polyol stabilized enzyme composition in such a manner that the composition looks attractive to the consumer, while simultaneously maintaining the shelf stability of the enzyme and of the composition. It has been found that polyacrylamides show a surprisingly fast hydration and quick gelling.

The acrylamide polymer to be used in the composition of the present invention is preferably substantially free of compounds or groups capable of binding Ca²⁺ ions or other divalent cations, such as for example COOH groups. As a consequence, the tendency to form a complex with calcium ions or other divalent cations will be negligible, while simultaneously minimizing the risk of destabilizing the enzyme. Divalent cations, in particular Ca²⁺, are added to enzyme compositions to stabilize the enzyme.

It is important in the present invention that the capacity of the acrylamide polymer to bind or chelate Ca²⁺ ions is as low as possible to ensure a sufficient stabilization of the enzyme. In this context, a low capacity of binding Ca²⁺ ions means that the capacity of the acrylamide polymer to bind Ca²⁺ ions is less than 80, preferably less than 20, more preferably less than 5 milligrams of Ca²⁺ ions per gram of acrylamide polymer. This is advantageous as the need to overcome unwanted complexation with calcium ions or other divalent cations needs not be compensated for through the addition of an extra amount of Ca⁺. A lower level of Ca²⁺ will thus suffice without this adversely affecting the stability of the enzyme. Lower Ca²⁺ levels are desirable in galenic compositions.

In the present invention, it is desirable to use acrylamide polymers that are as pure as possible where the monomer content has been minimized. Acrylamide monomers have been found to be toxic, therefore it is preferred to limit monomer concentration to below 2-5 ppm.

In addition to improving the shelf stability, the use of acrylamide polymers minimizes the risk of discoloration, as well as unwanted inhomogeneity, threadiness and tackyness of the composition, thus improving the cosmetic attractiveness of the composition.

The composition of the present invention preferably comprises 0.05 to 15 weight % of the acrylamide polymer. At a concentration below 0.05 wt. %, an insufficient thickening is oftentimes observed, whereas at a concentration above 15 wt. %, the viscosity gets too high. Preferred polyacrylamide concentrations range from 0.1-10 wt. %, more preferably 0.2-5 wt. % or 0.4-2.5 wt %, although 0.8-2 wt. % is a particularly preferred concentration range.

### DETAILED DESCRIPTION OF THE INVENTION

The polyacrylamide thickening agent of the present invention may optionally contain agents with additional thickening properties. Suitable examples of such additional thickening agents include hydrocolloids, such as xanthan, alginate, pectins; or modified cellulose, such as hydroxycellulose. It should however be understood that the main thickening agent of the present invention is the acrylamide polymer.

In a preferred embodiment of the present invention, the composition contains an amount of a reducing agent to protect the enzyme against destructive oxidation. Examples of suitable reducing agents for use in the composition of the instant invention include thiol compounds, for example dithiotreitol, or bet-mercaptoethanol, and an amino acid, for example methionine, or cysteine. Other suitable reducing agents which can be employed in the present invention include reducing agents without sulfury odors that belong to the well known group of antioxidants having an unsaturated aromatic ring with either an amine or a hydroxyl group, such as for example propyl gallate, various tocopherol derivatives, BHT and BHA. Suitable concentrations of the reducing compounds range from 0.1 to 100 millimoles/liter of the composition. To minimize sulfur odors from thiol compounds, the preferred concentrations range of the reducing compound is from 1 to 30 millimoles/liter of the composition. The preferred reducing agent is methionine since methionine is capable of releasing protease activity amino acids. Another preferred reducing agent is propyl gallate.

In another preferred embodiment of the present invention, the inventive composition additionally contains an amount of divalent cations, preferably Ca²⁺ ions, to further improve the stabilization of the enzyme. To guarantee adequate shelf stability, the divalent cations should preferably be present in concentrations higher than 0.02 millimoles/liter, more preferably higher than 1 millimole/liter of the composition. Moreover, the divalent cations are present in an amount which makes the cations readily available to the enzyme, yet not bound to another ingredient of the composition.

The polyol is preferably incorporated in the composition in a high concentration, i.e., a concentration which results in a sufficiently low water activity in the enzyme composition to adequately stabilize the enzyme. This means that the Aw of the composition is preferably lower than 0.9, more preferably lower than 0.85. It is known in the art that these concentrations may somewhat vary with the specific polyol used. Preferably, the polyol is used in a concentration of 20-95 wt. % with respect to the total weight of the composition, preferably in a concentration of 30-90%, more preferably in a concentration of 50-90%, in particular 60-80%. The low water activity of the composition thus obtained is advantageous for preventing bacterial outgrowth in the composition so that fungicide like propylparabens, which are often used in cosmetic applications, are adequate for total microbial preservation of the composition.

The type of the polyol that is used as the stabilization agent is not critical to the present invention. Thus, any polyol known to one skilled in the art that is capable of effectively stabilizing enzymes in aqueous compositions may be employed in the present invention. Particularly suitable polyols that may be employed include polyols selected from the group of glycerol, sorbitol, propylene glycol, butylene glycol, maltodextrins, or a sugar such as sucrose, lactose, glucose or trehalose. For topical applications, one should consider a polyol which is acceptable for topical use, i.e., glycerol, polyethylene glycol, butylene glycol, propylene glycol, trehalose or sorbitol.

The form in which the acrylamide polymer is incorporated into the composition may vary and is not essential to the present invention. The acrylamide polymer may for example be added in the form of an emulsion, an aqueous solution or even in a granular form that later on is dissolved.

In a preferred embodiment of the present invention, the acrylamide polymer is emulsified in a galenically acceptable carrier, for example isoparaffin. An example of such a product is SEPIGEL 305 (Seppic, Paris, France). Suitable concentrations of SEPIGEL 305 for viscosifying the polyol-stabilized enzyme composition range from 0.1-10 wt. %, 0.2 to 5.0%, preferably from 0.4 to 2.5%, more preferably from 0.8 to 2.0% (w/w).

Obviously the molecular weight of the polyacrylamide-based thickener used in the present invention is also an important parameter in establishing the final viscosity of the solution. Suitable preparations contain polyacrylamide molecules with molecular weights ranging from 1 to 40 million Daltons, preferably from 5 to 20 million Daltons.

The enzyme contained in the composition of the present invention may be any enzyme used in cosmetic compositions, for example belonging to the group of hydrolase, oxidoreductase, transferase or isomerase enzymes. More preferably, the enzyme is a protease, phosphatase, phytase, glycosidase, glucanase, mutanase (α-1,3-glucanase), dextranase, lysozyme, lipase, phospholipase, sulfatase, urease, glucose oxidase, peroxidase, lipoxygenase, superoxide dismutase, catalase, tyrosinase, transglutaminase, photolyase, a DNA repair enzyme such as T4 Endonuclase V or protein disulfide isomerase. The enzyme contained in the composition of the present invention may also be a mixture of two or more enzymes. The preferred enzyme is a protease enzyme.

The concentration in which the enzyme is present in the composition of the present invention will depend on the intended application, and will mostly vary from 10 mg to 10 g of pure protein per kg formulation. When used with the two compartment dispensing system described below, the enzyme composition is mixed with a second composition upon application. This mixing involves that the enzyme concentration is diluted, for example, by a factor 10, although this may vary with the intended application.

The present invention also envisages enzyme compositions in which the enzyme is formulated in particulate form.

The enzyme composition of the present invention is especially suitable for use in multi-compartment dispensing systems. In a preferred embodiment, the enzyme compositions of the instant invention are topically applied using the multi-compartment dispensing system disclosed in WO 97/27841. The dispensing system to be used is not critical to the present invention. Any dispensing system is contemplated which allows for the separate containment of the stabilized enzyme composition and the second composition. Separate containment is understood to include any form of separation enabling to prevent a substantial diffusion of water from one to the other composition. The multi-compartment dispensing system can be either a single dosage or a multi dosage dispensing system, for instance a dispensing system as referred to in U.S. Pat. No. 6,117,433.

The use of a multi-compartment dispensing system advantageously enables the simultaneous delivery of a stabilized enzyme composition and a second aqueous composition. Upon delivery, the stabilized enzyme composition and the aqueous composition are mixed, either in the dispensing system or in situ, resulting in a final, effective composition comprising the enzyme in an effective concentration and environment. The use of a multi-compartment dispensing system allows for the dilution of the stabilizing polyol present in the enzyme composition upon mixing with the second composition.

The dilution factor of the composition containing the enzyme into the second composition should be adequately chosen, i.e., taking into account the polyol concentration in the enzyme composition (and possibly the aqueous composition), so that the end concentration of the polyol does not preclude enzyme activity in the final composition. The dilution factor is determined by the ratio in which the enzyme composition and the second composition are delivered by the dispensing system. Preferably, the ratio between the enzyme composition and the second composition varies from 1:1 to 1:50, more preferably from 1:2 to 1:20, most preferably the ratio is 1:5 to 1:10.

When using a dispensing system, the concentration of the enzyme in the enzyme composition should be such that an effective enzyme concentration is reached in the final composition obtained upon mixing. The enzyme compositions of the invention are suitable for topical use, especially for use in cosmetics.

The following examples are given to illustrate the present invention as well as some advantages that can be obtained from using the same.

### EXAMPLE 1

### Cosmetic Properties of Glycerol Solutions Thickened with Various Compounds

In the first instance, tests were carried out in which various cosmetic grade thickeners were dissolved in a mixture consisting of 30% (w/w) water and 70% (w/w) glycerol only. Aim was to obtain a solution with a final viscosity not exceeding 5000 cP (shear rate 12.5 l/sec; Brookfield, spindle CS 4-29, 25° C., 50 rpm). Depending on the nature of the thickener and the recommendation of the supplier, individual thickeners were first dissolved in either pure water or pure glycerol, after which the other compound was added to obtain the final mixture. The pH of the mixture was adjusted to 5.5. Once prepared, the mixtures were stored for 2 weeks at ambient temperature whereupon the appearance and the cosmetic properties of the obtained gel were evaluated. Table 1 lists the various thickeners in their final concentration plus the cosmetic properties observed for the gels obtained.

TABLE 1

| Thickener and final concentration | Cosmetic properties of gel obtained |
|---|---|
| Carbopol Ultrez 10; 0.5% | Good |
| Carbopol ETD 2020; 0.5% | Good |
| Xanthan Gum; 0.5% | slime-like threads |
| Chitosan; 1% | malodorous |
| Hydroxyethylcellulose; 1% | slime-like threads, tends to form |
| | foam |
| Cetyl hydroxyethylcellulose; 1% | non-thickening, yellowish |
| Hydroxypropylmethylcellulose; 1% | non-homogeneous |
| Hectorite; 1% | non-thickening, brown |
| Mg-Al-silicate; 1% | non-thickening |
| Na-Mg-silicate; 3% in combination | good |
| with lactic acid to obtain pH 5.5 | |
| Polyvinylpyrrolidone (PVP) | non-thickening |
| MVE/MA copolymer crosslinked | non-thickening |
| with 1,9-decadiene | |
| Polyacrylamide emulsion; 1.5% | good |

As follows from the data presented in Table 1, only the two carbomer products (i.e., Carbopol Ultrez and Carbopol ETD 2020), the Na—Mg-silicate (i.e. Laponit XLG) in combination with lactic acid and the polyacrylamide emulsion (i.e. SEPIGEL 305) are capable of forming cosmetically acceptable gels with 70% glycerol in water.

### EXAMPLE 2

### Enzyme Stability in Cosmetically Acceptable Gels

According to the results obtained in Example 1 only a few thickeners are capable of forming cosmetically acceptable gels with water containing a high percentage of glycerol. In this Example, the inventors show the shelf stability of a protease in cosmetic gels in which different thickeners have been used to reach a viscosity of approx 3000 cP (shear rate 12.5/sec; Brookfield, spindle CS 4-29, 50 rpm, 25° C.).

The protease used is subtilisin (E.C. 3.4.21.62) purified from Maxatase R powder 2,16 BYU/kg as obtained from Genencor International, Brughes, Belgium. Subtilisin was purified according to the following protocol. Crude Maxatase powder was dissolved in 30 mmol/l sodium acetate pH 5.3 and the clear solution was applied to a strong cationic gel type resin (Sp Sepharose Fast Flow; Pharmacia). Upon elution of the column with 100 mmol/l sodium citrate pH 5.8, the peak representing the almost pure subtilisin was collected, pooled and lyophilized.

The lyophilized powder was dissolved in a small quantity of water and equally divided over five different formulations all containing 70% (w/w) glycerol, 0.02% (w/w) CaCl2.2 aq, 0.1% (w/w) methionine and 0.1% (w/w) propylparabens. To Formula 1 no thickener was added, whereas Formulae 2 to 4 were thickened to approx 5000 cP using Na—Mg-silicate (Laponit XLG, Laponite US) or carbomer (Carbopol ETD 2020 from BF Goodrich) or polyacrylamide (SEPIGEL 305 from Seppic) respectively. In all formulae, the pH was adjusted to a value between 5.0 and 5.5.

Subsequently each one of the five formulae was split into 4 portions. Three portions of the various formulae were carefully closed and stored at either −20° C., room temperature or in an incubator at 40° C.

The fourth portion was used immediately to determine the original proteolytic activity in each formula. This proteolytic activity was measured using a standard technique of hydrolysis of a synthetic chromogenic peptide substrate suitable for measuring subtilisin activities such as N-succinyl-Ala-Ala-Pro-Phe-pNA (from Sigma). Enzymatic hydrolysis was followed at 25° C., in PBS buffer pH 7.2 and with a substrate concentration of 1 millimol/liter by the optical extinction at 410 nanometer. The initial rates of hydrolysis were used to quantify the proteolytic activities present in the various formulae. Then the other three portions of the various formulae were carefully closed and stored at either −20° C., room temperature or in an incubator at 40° C.

After 6 weeks of incubation, the remaining proteolytic activity in all formulae and in all portions (−20° C., room temperature and 40° C.) was measured using exactly the same activity test. Only in the formula without thickener added and in the formula thickened with polyacrylamide (i.e. SEPIGEL 305) a less than 20% deterioration of the original proteoytic activity could be established. Remarkable was that the polyacrylamide thickened formulae exhibited minor differences only in the proteolytic activities as established in the samples kept at the −20° C., room temperature and 40° C. regimes, implying excellent enzyme stabilities under all conditions tested.

In the other formulae thickened with either the Na—Mg-silicate or the carbomer product, the proteolytic activity was found to be seriously deteriorated after the 6 weeks storage period, i.e., to levels below 50% of the original activity values. Quite surprising was that in these samples all three temperature regimes showed such inadequate stabilities.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the spirit and scope of the appended claims.

1. A composition comprising at least one enzyme, a polyol for stabilizing the enzyme and an acrylamide polymer thickening agent having polymer units of the formula —(CH₂CR(CONH₂)—, in which R is hydrogen or a methyl group.

2. The composition of claim 1 wherein the —(CH₂CR(CONH₂)— polymer in the composition has a concentration of from 0.05 to 15 wt. % with respect to the total weight of the composition.

3. The composition of claim 2 wherein the concentration of the —(CH₂CR(CONH₂)— polymer in the composition is from 0.1 to 10 wt. % with respect to the total weight of the composition.

4. The composition of claim 2 wherein the concentration of the —(CH₂CR(CONH₂)— polymer in the composition is from 0.2 to 5 wt. % with respect to the total weight of the composition.

5. The composition of claim 2 wherein the concentration of the —(CH₂CR(CONH₂)— polymer in the composition is from 0.8 to 2 wt. % with respect to the total weight of the composition.

6. The composition of claim 1 wherein the —(CH₂CR(CONH₂)— polymer comprises less than 5 ppm of an acrylamide monomer.

7. The composition of claim 1 wherein the composition has a water activity of below 0.9.

8. The composition of claim 1 wherein the composition has a water activity below 0.85.

9. The composition of claim 1 wherein the —(CH₂CR(CONH₂)— polymer has a divalent ion binding capacity of less than 80 mg/g of polymer.

10. The composition of claim 1 wherein the —(CH₂CR(CONH₂)— polymer has a divalent ion binding capacity of less than 20 mg/g of polymer.

11. The composition of claim 1 wherein the —(CH₂CR(CONH₂)— polymer has a divalent ion binding capacity of less than 5 mg/g of polymer

12. The composition of claim 1 further comprising a reducing agent.

13. The composition of claim 12 wherein the reducing agent is methione, cysteine, or antioxidants having an unsaturated aromatic ring with an amine or a hydroxyl group.

14. The composition of claim 1 wherein the composition comprises at least 0.02 millimole/liter of divalent cations.

15. The composition of claim 14 wherein the divalent cations are Ca²⁺ ions.

16. The composition of claim 1 wherein the composition comprises at least 1 millimole/liter of divalent cations.

17. The composition of claim 1 wherein the polyol is present in a concentration of 20-95 wt % with respect to the total weight of the composition.

18. The composition of claim 1 wherein the polyol is present in a concentration of 30-90 wt. % with respect to the total weight of the composition.

19. The composition of claim 1 wherein the polyol is present in a concentration of 60-80 wt. % with respect to the total weight of the composition.

20. A multi-compartment dispensing system comprising a first compartment comprising a stabilized enzyme composition, and a second compartment comprising an aqueous composition for activating the enzyme, wherein said stabilized enzyme composition comprises at least one enzyme, a polyol for stabilizing the enzyme and an acrylamide polymer thickening agent having polymer units of the formula —(CH₂CR(CONH₂)—, in which R is hydrogen or a methyl group.
